(19) United States
(12) Patent Application Publication
(10) Pub. No.: US 2003/0013692 A1
(43) Pub. Date: Jan. 16, 2003

(54) Methods of treating neurological disorders

(76) Inventors: Steven R. Gullans, Natick, MA (US); Satinder Sarang, Boston, MA (US)

Correspondence Address: MINTZ, LEVIN, COHN, FERRIS,, GLOVSKY and POPEO, P.C., One Financial Center, Boston, MA, 02111, US

(21) Appl. No.: 10/052,691

(22) Filed: Jan. 18, 2002

Related U.S. Application Data
(60) Provisional application No. 60/262,720, filed on Jan. 19, 2001.

(51) Int. Cl. A61K 31/573

(52) U.S. Cl. 514/179

(57) ABSTRACT

The invention features a method for inhibiting neuronal cell death in a mammal by administering to the mammal a cytoprotective composition.

### RELATED U.S. APPLICATION

This application claims priority to U.S. Ser. No. 60/262,720 filed Jan. 19, 2001, which is incorporated herein by reference in its entirety.

### FIELD OF THE INVENTION

The invention relates to methods of treating neurological disorders.

### SUMMARY OF THE INVENTION

The invention features methods of inhibiting death of a neuronal cell in a mammal by administering to the mammal a cytoprotective composition. A cytoprotective compound is one that inhibits cell death. Preferably, the compound function to inhibit oxidative stress-induced death of neuronal cells. The compounds are safe for human administration and, in some cases, have been administered patients to treat non-neuronal indications

A neuronal cell is any cell derived from the central or peripheral nervous system, e.g., neuron, neurite or dendrite.

To determine whether a compound inhibits oxidative-stress induced cell death, a candidate compound is tested by incubating the compound with a primary or immortalized neuronal cell (e.g., SH-SY5Y), inducing a state of oxidative stress of the cells (e.g., by incubating them with H₂O₂) and measuring cell viability is measured using standard methods. As a control the cells are incubated in the absence if the candidate compound and then the treated cells are incubated in the absence of the candidate compound and then treated to induce a state of oxidative stress. A decrease in cell death (or an increase in the number of viable cells) in the compound treated sample indicates that the compound inhibits oxidative-stress induced cell death. The test is repeated using different does of the compound to determine the dose range in which the compound functions to inhibit oxidative-stress induced cell death.

A steroid compound is administered to inhibit neuronal cell death. The steroid is administered at a dose sufficient to inhibit oxidative stress-induced neuronal cell death. Oxidative stress-induced cell death occurs after neuronal cells are deprived of oxygen, e.g., as a result of a progressive neurodegenerative condition or an acute episode such as a stroke or exposure to a toxic compound.

Alternatively, the composition is administered at a dose sufficient to inhibit apoptotic death of the neuronal cell. The compositions preferentially inhibits apoptotic death compared to necrotic death of the cell. Cytotoxicity or cell death may occur by either necrosis or apoptosis. Necrosis, which is not genetically controlled, is usually the result of physical or chemical injury. Apoptosis is genetically controlled and is a cellular response to a specific stimuli, e.g., a cell surface-generated signal. Necrosis involves the destruction of cytoplasmic organelles and a loss of plasma membrane integrity, whereas cells undergoing apoptosis exhibit cell shrinkage, membrane blebbing, chromatin condensation and fragmentation. After the DNA damage in the caspase enzyme pathway, there are a series of events which occur that involve calcium activation and calpain enzymes which further leads to other cellular changes and regulation of cytoplasmic enzymes. For example, the steroid compound is a progestin compound such as (17α)-17-Hydroxy-19-norpregn-4-en-20-yn-3-one or 17a-(acetyloxy)-6-methylpregna-4,6-diene-3,20-dione. An anti-inflammatory steroid such as flunisolide is administered to inhibit neuronal cell death. Such as steroid is administered at a dose which inhibits oxidative stress-induced cell death with or without anti-inflammatory effects.

The mammal to be treated with the compounds discussed herein is suffering from or at risk of developing a neurological disorder such as diabetic neuropathy, cerebral hypoxia, encephalitis and menengitis. For example, the mammal is at risk of experiencing a stroke or has suffered a stroke. In another example, the mammal is suffering from or at risk of developing neurodegenerative disorder such as Amyotrophic Lateral Sclerosis, Alzheimer's disease, Huntington's disease and Parkinson's disease. The methods are suitable for treating human patients as well as non-human animals such as livestock or pets (e.g., dogs or cats).

The composition to be administered contains an anti-motion sickness agent. Preferably, the anti-motion sickness agent is administered at a dose sufficient to inhibit oxidative stress-induced neuronal cell death or at a dose sufficient to inhibit apoptotic death of neuronal cells. Anti-motion sickness agents to be administered include H1 histamine receptor blocker compounds such as 1-[(4-Chlorophenyl)phenylmethyl]-4-[(3-methylphenyl)methyl]piperazine and belladonna alkaloids such as 6β,7β-epoxy-1αH,5αH-tropan-3α-ol(−)-tropate.

Antibiotic compounds are administered at a dose sufficient to inhibit oxidative stress-induced neuronal cell death or at a dose sufficient to inhibit apoptotic death of neuronal cells. For example, the antibiotic compound is a macrolide such as erythromycin, troleandomycin, azithromycin or clarithromycin. Tetracycline compounds or derivative thereof (e.g. chlorotetracycline, oxytetracycline, demeclocycline, methacycline. doxycycline and minocycline) are also administered to inhibit neuronal cell death. Other antibiotics such as tobramycin compounds or sulfacetamide compounds are also suitable as cytoprotective compounds.

The methods include inhibiting neuronal cell death by administering a calcium channel blocker compound such as isopropyl (2-methoxyethyl) 1,4-dihydro-2,6-dimethyl-4-(3-nitrophenyl)-3,5-pyridine-dicarboxylate; α-[3-[[2-(3,4-dimethoxyphenyl)ethyl]methylamino]propyl]-3,4-dimethoxy-α-1(1-methylethyl)benzeneacetonitrile, 3,5-pyridinedicarboxylic acid; 1,4-dihydro-2,6-dimethyl-4-(2-nitrophenyl)-, dimethyl ester 1,8-dihydroxy-9(10H)-anthracenone. The compounds are administered as doses which inhibit oxidative stress-induced neuronal cell death or at doses which inhibit apoptotic cell death.

Anti-depressant compounds such as lithium carbonate, trazodone, bupropion hydrochloride, fluoxetine hydrocloride and sertraline hydrochloride and alkali metal compositions, e.g., those which contain lithium, caesium, rubidium and francium, are also used to inhibit neuronal cell death. Other compounds including anti-arrhythmic agents such as a beta-adrenergic receptor blocking compound (e.g., 1-N-[4-[1-hydroxy-2[(methylethyl)amino]ethyl]phenyl]methane-sulfonamide monohydrochloride and (S)-1-[(1,1-dimethylethyl)amino]-3-[[4-(4-morpholinyl)-1,2,5-thiadiazol-3-yl]oxy]-2-propanol (Z)-2-butenedioate) or sodium channel blockers (e.g., lidocaine, mexiletine and prilocaine) are also used in the methods described herein. Certain vitamins, minerals, and herbal compounds such as yohimbine, zinc, α-carotene, docosahexaenoic acid, retinol acetate, and presynaptic alpha-adrenergic receptor blocking compounds (e.g., yohimbine, medetomidine hydrochloride and atipamezole) inhibit neuronal cell death. Muscle relaxant compounds such as (Z)-5-fluoro-2-methyl-1-[[p-(methylsulfyl)phenyl]methylene]-1 H-indene-3 acetic acid and dopaminergic agonists also inhibit oxidative stress-induced neuronal cell death. For example, a suitable dopaminergic agonist is a prolatin-inhibiting compound such as bromocriptine.

Other cytoprotective compounds include carbonic anhydrase inhibitors (e.g., methazolamide, acetazolamide, dorzolamide and brinzolamide), anesthetic compounds (e.g., a dyclonine hydrochoride and corticosteroids such as pramoxine, hydocortizone, hetamethazone, budesonide, prednisone and cortisone), opioid antagonists (e.g., naltrexone, propoxyphene and pentazocine), thiol compounds (e.g., 2-mercaptoethanesulfonic acid, propyl mercaptan, ethyl mercaptan and butyl mercaptan), non-steroidal anti-inflammatory compounds (e.g., sulindac, ibuprofen, nabumentone, naproxen and acetaminophen).

Although the compounds described herein have been used clinically to treat a number of diseases, the cytoprotective activity with respect to neurons was surprising.

Other features and advantages of the invention will be apparent from the following description of the preferred embodiments thereof, and from the claims.

### DETAILED DESCRIPTION

Oxidative stress and the resulting death of neurons is a major pathological factor involved in the progression of numerous neurodegenerative diseases including Amyotrophic Lateral Sclerosis, Parkinson's and Alzheimer's disease, and stroke. A panel of FDA approved drugs was screened, and drugs, which prevent neuroblastoma cells from dying from oxidative stress, were identified.

Identification of Cytoprotective Drugs

A drug library of approximately 1,400 FDA approved drugs was screened for cytoprotective activity. Neuroblastoma cells (SH-SY5Y; ATCC No: CRL-2266) were cultured in 96 well plates and then incubated with the 100 μM of a drug for 24 hr. The drug was removed from SH-SY5Y cells, and the cells washed with phosphate buffered saline. The cells were then incubated with 6 mM hydrogen peroxide for 4 hr and cell viability assayed using a fluorescent probe (calcein-AM). Fluorescence was measured using an LJL Analyst fluorescence plate reader. From the initial screen of 1000 compounds, 34 drugs were found to be cytoprotective. By cytoprotective is meant that the drug decreases the level of oxidative stress-induced neuronal death in a population of drug-treated cells compared to the level observed in a population of cells that were not contacted with the drug.

Dose response experiments were then carried out to determine the dose range of the cytoprotective activity. Generally, the drugs identified in screen were 60% to 98% cytoprotective over a dose range of 1 μM to 100 μM, respectively. These data indicate that the screening method described above reliably identified drugs, which decrease the level of oxidative stress-induced cell death.

Characterization of Cytoprotective Drugs

Drugs identified in the primary screen described above are further evaluated for cytoprotective activity using other neuronal cell lines and neuronal tissues. Examples of neuronal cell lines are the primary neuronal cell lines HCN-1A (ATCC No: CRL 10442), HCN-2 (ATCC No: CRL 10742), PC-12 (ATCC No: CRL 1721), and PC-12 expressing hSOD1.

Similar to the initial evaluation, the neural cell lines or tissues are cultured in 384 and 96-well plates and the cells or tissue incubated with the 100 μM of a drugs for 24 hr. The drugs are optionally removed from the cells, e.g., by washing with phosphate buffered saline. The cells are then incubated with 6 mM hydrogen peroxide for 4 hr and cell viability assayed using a fluorescent probe (calcein-AM). Fluorescence is measured using an LJL Analyst fluorescence plate reader. An increase in cell viability in drug treated cells treated compared to untreated cells indicates that the test agent is cytoprotective. To evaluate apoptotic cell death, cells are incubated in the presence or the absence of a drug, and the apoptosis measured using known methods in the art (e.g., electrophoresis or caspase-based assays). Optionally, an oxidative stress state is induced prior to measuring apoptotic cell death in treated cells compared to untreated cells indicates that the drug inhibits apoptotic cell death.

Other indices are used to evaluate the cytoprotective activity of the identified compounds. These indices include for example, caspase activation assays (marker for cellular apoptosis), measurement of mitochondrial membrane potential (energy production), and cell membrane integrity. All these cell based assays uses a florescent probe. Fluorescence is measured using an LJL Analyst fluorescence plate reader.

Elucidation of the Molecular Mechanism of Cytoprotective Activity

To elucidate the molecular mechanisms involved in cytoprotective activity, neural cell lines are transfected with genes and transcriptional elements that are thought to be involved in the progression of numerous neurological disorders. The genes and transcriptional elements are fused with reporter genes such as green fluorescent protein (GFP), red fluorescent protein (RFP), and luciferase. These genes and transcriptional elements are fused with reporter genes that of the genes and transcriptional elements are monitored following exposure of the cell to the cytoprotective agents.

Determination of the Genes that Confer Cytoprotection.

To determine which genes confer cytoprotection to the cells, gene expression profiles of cells exposed to oxidant stress and the drug are compared to the gene expression profiles of similar cells exposed to oxidant stress but not exposed to the drug. Difference in genes expression in the cells that are exposed to the drug as compared to cells not exposed to the drug indicate that the gene may confer cytotoprotection. Gene expression is measured using oligonucleotides and cDNA microarrays

Determination Cytoprotection in Vivo

Numerous animals models for neurological disorders are known in the art. These models are used to study the cytoprotective activity of the drugs in vivo. A compound is cytoprotective for a particular neurological disorder when the subject displays fewer symptoms associated with the neurological disease in the presence of the compound compared to the symptoms exhibited in the absence of the compound.

For example a model for ALS is a, transgenic mice expressing multiple copies of a mutated cytosolic Cu/Zn superoxide dismutase (SOD1) gene develop an ALS-like motoneuron disorder. (Jaarma et al., 2000 Neurobiol Dis. 7(6): 623-43)

The gene responsible for Huntington's disease, IT15 has been identified. A rodent model for Huntington's disease is described in Broullet, et al. (Broullet et al., 1999 Prog Neurobiol. 59(5): 427-68.

Animal models to study Parkinson's disease have been developed in a number of species by toxin induced and genetic experimental models. For example, rats treated with the 6-hydroxydopamine replicate the neurochemical, morpholoic and behavioral changes seen in humans with Parkinson's disease. (Tolwani et al., 1999 Lab Anim. Sci. 49(4):363-71.

Models for other neurological disorders are also known. See generally, Isozumi, et al 1998 J Exp Clin Med 23(3):103-17 (models for cerebral ischemia); Muhlestein, 2000 J. Infect. Dis. (181) (Suppl 3):S505-7 (rabbit arthersclerosis model); and Hounsom, et al 1997 Clin Neurosci: 4(6):380-9 (neuropathy animal models).

Other models include ALS mice models, Parkinson's disease fly model, for Stroke and the rat and mouse Stroke Injury model.

Methods of Diagnosing a Neurological Disorder or a Predisposition to Developing a Neurological Disorder

Neurological disorders, include neurodegenerative disorders such as Amyotrphic Lateral Sclerosis, Alzheimer's disease, Huntington's disease and Parkinson's disease. Neurodegenerative diseases are characterized by gradual progressive neuronal cell death occurring for reasons that are largely unknown. Other neurological disorders include neuropathy, e.g., diabetic neuropathy, encephalitis and menengitis. A neurological disorder also includes stroke and cerebral hypoxia. Stroke results in neuronal cell death due to diminished blood flow to the brain. In contrast, cerebral hypoxia results in neuronal cell death due to diminished the oxygen supply to the brain.

Neurological disorders are diagnosed, typically by a physician using standard methodologies known be those skilled in the art. Such methods include, neurologic history, neurological examination. Neurological examination is accomplished by a systematic physical examination of all functions of the cerebrum, peripheral nerves and muscle. Diagnosis is also made using techniques for imaging the nervous system with such as computed tomography, magnetic resonance imaging, myelography, and positron emission tomography.

Amyotrophic Lateral Sclerosis

Amyotrophic lateral sclerosis (ALS), often referred to as “Lou Gehrig's disease,” is a progressive neurodegenerative disease that attacks nerve cells in the brain and the spinal cord. The progressive degeneration of the motor neurons in ALS eventually lead to their death. As the motor neurons die, the ability of the brain to initiate and control muscle movement is lost.

ALS typically develops in individuals who are between the ages of 40 and 70, with an average age of 55 at the time of diagnosis.

There are three types of ALS, Guamanian, familial and sporadic. Sporadic is the most common type, and to date has not been correlated with any risk factors. In contrast, approximately 10% of individuals diagnosed with ALS have a genetic predisposition characterized by mutation in the cytosolic Cu/Zn superoxide dismutase (SOD1) gene. Guanmanian ALS is found in a large population of individuals from Chamorros of the Mariana Islands.

Early symptoms of ALS include the muscle weakness in the hands, arms, legs or the muscles of speech, swallowing or breathing, twitching (fasciculation) and cramping of muscles, especially those in the hands and feet, impairment of the use of the arms and legs, “thick speech” and difficulty in projecting the voice. As the disease progresses limbs begin to look “thinner” as muscle tissue atrophies.

The diagnosis of ALS includes determining the presence of (1) evidence of lower motor neuron (LMN) degeneration by clinical, electrophysiological or neuropathologic examination, (2) evidence of upper motor neuron (UMN) degeneration by clinical examination, and (3) progressive spread of symptoms or signs within a region or to other regions, as determined by history or examination, together with the absence of electrophysiological and pathological evidence of other disease processes that might explain the signs of LMN and/or UMN degeneration, and neuroimaging evidence of other disease processes that might explain the observed clinical and electrophysiological signs. El Escorial World Federation of Neurology criteria for the diagnosis of amyotrophic lateral sclerosis, Journal of the Neurological Sciences 124: 96-107.

Parkinson's Disease

Parkinson's Disease is a neurodegenerative disease that manifests as a tremor, muscular stiffness and difficulty with balance and walking. A classic pathological feature of the disease is the presence of an inclusion body, called the Lewy body, in many regions of the brain. Risk factors, such as rural living, farming, drinking well water, being exposed to industrial chemicals, herbicides and insecticides can also be considered in the early diagnosis of Parkinson's disease.

Until relatively recently, Parkinson disease was not though to be heritable, and research was primarily focused on environmental risk factors such as viral infection or neurotoxins. Severe Parkinson's-like symptoms have been described in people who took an illegal drug contaminated with the chemical MPTP (1-methyl-4-phenyl-1,2,3,6-tetrahydropyridine) and in people who suffered a particularly severe form of influenza during an epidemic in the early 1900s. However, a positive family history was gradually perceived to be a risk factor, a view that was confirmed last year when a candidate gene for some cases of Parkinson disease was mapped to chromosome 4. Mutations in this gene have now been linked to several Parkinson disease families. The product of this gene, a protein called alpha-synuclein.

The first symptom of Parkinson's disease is tremor (trembling or shaking) of a limb, especially when the body is at rest. The tremor often begins on one side of the body, frequently in one hand. Other common symptoms include slow movement (bradykinesia), an inability to move (akinesia), rigid limbs, a shuffling gait, and a stooped posture. People with Parkinson's disease often show reduced facial expression and speak in a soft voice. Occasionally the disease also causes depression, personality changes, dementia, sleep disturbances, speech impairments, or sexual difficulties. The symptoms first appear, on average, at about age 60, and the severity of Parkinson's symptoms tends to worsen over time

The diagnosis is based on a neurological examination, which includes evaluation of symptoms and their severity. When symptoms are significant, a trial test of drugs (primarily levodopa [L-dopa]) may be used to further diagnose the presence of PD. If a patient fails to benefit from levodopa, a diagnosis of Parkinson's disease is questionable. Computed tomography (CT) or magnetic resonance imaging (MRI) scans of the brain may be used to help rule out other diseases with symptoms that resemble PD.

Huntington's Disease

In Huntington's disease (HD), is characterized by an uncontrollable involuntary movements, psychiatric abnormalities and a loss of intellectual functions (dementia). Involuntary movements, such as chorea, result from abnormalities in the structures called basal ganglia which are located deep in the brain and regulate motor movements. One of these structures called striatum shows a decreased volume in HD. The atrophy is due to degeneration of a particular subpopulation of the neurons (brain cells with electrical activities) called medium-size spiny neurons located within the striatum. Dementia and psychiatric abnormalities are due to degeneration of neurons outside the basal ganglia. A loss of neurons in the cerebral cortex (the surface layers of the brain) is particularly prominent in HD.

The mechanism of the degeneration is not fully understood. However, the final process of brain cell death appears to be mediated by a class of amino acids (called excitatory amino acids) released from other neurons in which excessive excitation of neurons causes “exhaustion” of the neurons and eventually leads to cell death, especially when the neurons already suffer from a disease process. This phenomenon is called “excitotoxic cell death.”

About 10% of HD cases have their onset before age 20, but the typical peak age at onset is in the 4th and 5th decade. Young-onset patients usually inherit the disease from their father while older-onset patients are more likely to inherit the gene from their mother. Juvenile HD (onset of symptoms before 20 years) typically presents with the combination of progressive parkinsonism, dementia, ataxia, and seizures. In contrast, adult HD usually presents with the insidious onset of clumsiness and adventitious movements which may be wrongly attributed to simple nervousness. Slowness of movement (bradykinesia) is usually evident in patients with the rigid form of HD, but when it coexists with chorea it may not be fully appreciated on a routine examination. While bradykinesia is most pronounced in the rigid-akinetic patients, it is also evident in patients with the typical choreic variety of HD. When bradykinesia predominates, the patients exhibit parkinsonian findings some of which may be subtle. Micrographia may be one manifestation of underlying parkinsonism; when chorea predominates the handwriting is characterized by macrographia. Bradykinesia in HD may be an expression of “post-synaptic parkinsonism” and possibly explains why a reduction in chorea with anti-dopaminergic drugs rarely improves overall motor functioning and indeed may cause an exacerbation of the motor impairment.

Alzheimer's Disease

Alzheimer's Disease (AD), is the most common cause of dementia in the elderly, is a heterogeneous group of neurodegenerative disorders. The incidence rate for dementia in general is 187 new cases/100,000 population/year, and for AD it is 123 new cases/100,000 population/year. Males and females are affected about equally.

The main risk factor for Alzheimer's disease is increased age. The rates of the disease increase markedly with advancing age, with 25 percent of people over 85 suffering from Alzheimer's or other severe dementia. A genetic basis has been identified through the discovery of several genetic markers on chromosomes 21 and 14 for a small subgroup of families in which the disease has frequently occurred at relatively early ages (beginning before age 50). Genetic markers on chromosome 14 include the genes for Presenilin 1 and Presenilin. Markers for AZ on chromosome 21 include the gene for Aβ precursor protein. In addition some evidence points to chromosome 19, specifically the apolipoprotein E gene is implicated in certain other families that have frequently had the disease develop at later ages.

The end-stage AD brain shows diffuse cerebral atrophy with enlarged ventricles, narrowed cortical gyri and widened sulci. These changes are attributed to neuronal loss. While the loss of neurons in AD generally exceeds that seen during normal aging, there may be overlap between the AD brain and the brains of age matched normal subjects. However, individual neuronal groups in neurodegenerative disorders and normal aging vary in their susceptibility for degeneration. Specifically, the hippocampal formation is consistently and heavily involved in the pathology of AD, and considerably less affected in normal aging.

The definitive diagnosis of Alzheimer's disease can only be made by microscopic examination of the brain. The end-stage AD brain shows diffuse cerebral atrophy with enlarged ventricles, narrowed cortical gyri and widened sulci. These changes are attributed to neuronal loss. While the loss of neurons in AD generally exceeds that seen during normal aging, there may be overlap between the AD brain and the brains of age matched normal subjects. However, individual neuronal groups in neurodegenerative disorders and normal aging vary in their susceptibility for degeneration. Specifically, the hippocampal formation is consistently and heavily involved in the pathology of AD, and considerably less affected in normal aging.

The accuracy of the clinical diagnosis however, can be as high as 90%. Since symptomatic presentation may vary, the physician should suspect dementia when seeing a patient with memory or intellectual dysfunction, psychiatric symptoms or physical complaints (or both) that do not fit a discernible pattern of other organic diseases. The clinical diagnosis of probable Alzheimer's Disease rests on a gradually progressive problem with memory and at least one other cognitive function in addition to physical, neurological, and laboratory tests unrevealing of evidence for an alternate medical or neurological disease as the cause.

In addition to medical history, physical and neurological examination other diagnostic measure includes serum glucose levels, erythrocyte sedimentation rate, heavy metal screens, if history of exposure known or suspected, tests for human immunodeficiency antibodies, urinalysis, chest roentgenogram, electrocardiogram (EKG), electroencephalography (EEG), cerebrospinal fluid analysis to rule out chronic infections (i.e., cryptococcosis) or lymphomas, and SPECT, a blood flow study useful to distinguish vascular from degenerative dementias.

Diabetic Neuropathy

Diabetic neuropathy is a nerve disorder caused by diabetes. Symptoms of neuropathy include numbness and sometimes pain in the hands, feet, or legs. Nerve damage caused by diabetes can also lead to problems with internal organs such as the digestive tract, heart, and sexual organs, causing indigestion, diarrhea or constipation, dizziness, bladder infections, and impotence. In some cases, neuropathy can flare up suddenly, causing weakness and weight loss.

Diagnoses neuropathy based on symptoms and a physical exam. During the exam, the doctor may check muscle strength, reflexes, and sensitivity to position, vibration, temperature, and light touch. A simple screening test to check point sensation in the feet can be done. The test uses a nylon filament mounted on a small wand. The filament delivers a standardized 10-gram force when touched to areas of the foot. Patients who cannot sense pressure from the filament have lost protective sensation and are at risk for developing neuropathic foot ulcers.

Nerve conduction studies can be used to determine the flow of electrical current through a nerve. Impulses that seem slower or weaker than usual indicate possible damage to the nerve. Electromyography (EMG) can be used to see how well muscles respond to electrical impulses transmitted by nearby nerves. screen. A response that is slower or weaker than usual suggests damage to the nerve or muscle.

Exemplary Cytoprotective Compounds

Exemplary cytoprotective steroids include progestin compounds such as, norethindrone, and megestrol and dithranol. Norethindrone ((17α)-17-Hydroxy-19-norpregn-4-en-20-yn-3-one) is a oral contraceptive containing only progestin used to prevent conception by suppressing ovulation. Megestrol (17a-(acetyloxy)-6-methylpregna-4,6-diene-3,20-dione) is a progestin compound with antineoplastic effects against cancers as such as endometrial carcinoma, breast carcinoma. Pharmacologic doses of megestrol acetate decrease the number of hormone-dependent human breast cancer cells and modulates the stimulatory effects of estrogen on these cells. Dithranol (1,8-Dihydroxy-9(10H)-anthracenone) is a steroid compound with ntipsoriatic antifungal action. Other exemplary steroid compounds anti-inflammatory steroid compounds include Flunisolide (6α-fluoro-11β,16α,17,21 tetrahydroxy-pregna-1,4-diene-3,20-dione cyclic 16,17 acetal with acetone).

Exemplary cytoprotective motion sickness compounds include meclizine and scoplamine. Meclizine (1-[(4-Chlorophenyl)phenylmethyl]-4-[(3-methylphenyl)methyl]piperazine) is an antiemetic H 1 histamine receptor blocker. Scopolamine (6β,7β-epoxy-1αH,5αH-tropan-3α-ol(−)-tropate) is a belladonna alkaloid. The drug has a long history of oral and parenteral use for central anticholinergic activity, including prophylaxis of motion sickness.

Exemplary cytoprotective antibiotic compounds include macrolide antibiotic compounds such as erythromycin, troleandomycin a synthetic acetylated ester of oleandomycin, azithromycin and clarithrmycin. Other exemplary antibiotic compounds includes tetracycline and tetracycline derivatives such as chlortetracycline, oxytetracycline, demecyline, methacycline and minocycline. Exemplary antibiotic compounds also include aminoglycoside antibiotic compounds such as tobramycin. Exemplary antibiotic compounds further include kanamycin, tobramysin and sulfacetamide.

Exemplary cytoprotective calcium channel blocker compounds include Nimodipine, Dithranol, Verapamil and Nifedipine. Nimodipine (Isopropyl (2-methoxyethyl) 1,4-dihydro-2,6-dimethyl-4-(3-nitrophenyl)-3,5-pyridine-dicarboxylate) inhibits calcium ion transfer into cells and inhibits contractions of vascular smooth muscle. It is highly lipophilic, allowing it to cross the blood-brain barrier; concentrations of nimodipine as high as 12.5 ng/mL have been detected in the cerebrospinal fluid of nimodipine treated subarachnoid hemorrhage (SAH) patients. Dithranol (1,8-Dihydroxy-9(10H)-anthracenone), electively inhibits calcium ion influx across the cell membrane of vascular smooth muscle and cardiac muscle without altering serum calcium concentrations. It is a peripheral arterial vasodilator which acts directly on vascular smooth muscle. The binding of nifedipine to voltage-dependent and possibly receptor-operated channels in vascular smooth muscle results in an inhibition of calcium influx through these channels. Verapamil, (α-[3-[[2-(3,4-Dimethoxyphenyl)ethyl]methylamino]propyl]-3,4-dimethoxy-α-1(1-methylethyl)benzeneacetonitrile) is a calcium channel blocker that exerts its pharmacologic effects by modulating the influx of ionic calcium across the cell membrane of the arterial smooth muscle as well as in conductile and contractile myocardial cells. Verapamil exerts antihypertensive effects by decreasing systemic vascular resistance, usually without orthostatic decreases in blood pressure or reflex tachycardia. Verapamil does not alter total serum calcium levels. Nifedipine (3,5-pyridinedicarboxylic acid, 1,4-dihydro-2,6-dimethyl-4-(2-nitrophenyl)-, dimethyl ester) is used in the management of vasospastic angina.

Exemplary cytoprotective anti-depressant compounds include lithium carbonate, trazodone, buproin hydrochlorides, fluxetine hydochloride and sertraline hydrochloride. Lithium alters sodium transport in nerve and muscle cells and effects a shift toward intraneuronal metabolism of catecholamines. Trazodone (1-[3-[4-(3-chlorophenyl)-1-piperazinyl]propyl]-1,2,4-triazolo[4,3-a]pyridin-3(2H)-one monohydrochloride) is an antidepressant chemically unrelated to tricyclic, tetracyclic, or other known antidepressant agents. In animals, trazodone selectively inhibits serotonin uptake by brain synaptosomes and potentiates the behavioral changes induced by the serotonin precursor, 5-hydroxytryptophan.

Exemplary cytoprotective alkali metal compounds include lithium, caesium, rubidium and francium.

Exemplary cytoprotective antiaarhythimic compounds include beta-adrenrgic receptor blocking compounds such as timolol maleate and solatol. Timolol Maleate ((S)-1-[(1,1-dimethylethyl)amino]-3-[[4-(4-morpholinyl)-1,2,5-thiadiazol-3-yl]oxy]-2-propanol (Z)-2-butenedioate (1:1) salt) is a beta 1 and beta 2 (non-selective) adrenergic receptor blocking agent that does not have significant intrinsic sympathomimetic, direct myocardial depressant, or local anesthetic activity. Timolol maleate decreases the positive chronotropic, positive inotropic, bronchodilator, and vasodilator responses caused by beta-adrenergic receptor agonists. Sotalol (, 1-N-[4-[1-hydroxy-2-[(methylethyl)amino]ethyl]phenyl]methane-sulfonamide monohydrochloride) is has Class II (beta-adrenoreceptor blocking) and Class III (cardiac action potential duration prolongation) properties. Sotalol hydrochloride is a racemic mixture of d-and l-sotalol. The beta-blocking effect of sotalol is non-cardioselective, half maximal at about 80 mg/day and maximal at doses between 320 and 640 mg/day. Class III effects are seen only at daily doses of 160 mg and above. Sotalol hydrochloride prolongs the plateau phase of the cardiac action potential in the isolated myocyte, as well as in isolated tissue preparations of ventricular or atrial muscle. Other exemplary antiarrhythmic compounds of the invention include, sodium channel blocker compounds such as lidocaine, mexiletine and prilocaine. Mexiletine (1-methyl-2-(2,6-xylyloxy)-ethylamine hydrochloride) is structurally similar to lidocaine, but orally active.

Exemplary cytoprotective dietary supplement compounds include yohimbine, zinc, β-caroten, dososahexaenoic acid omega-3 oil (DHA-250) and retinal acetate. Yohimbine, blocks presynaptic alpha-2 adrenergic receptors. Its action on peripheral blood vessels resembles that of reserpine, though it is weaker and of short duration. Yohimbine' peripheral autonomic nervous system effect is to increase parasympathetic (cholinergic) and decrease sympathetic (adrenergic) activity. Zinc has recently been the subject of renewed research interest because of epidemiological evidence indicating an inverse relationship between intake of carotenoids-rich plant substances and risk of certain cancers. Docosahexaenoic acid. Retinol acetate is a Vitamin precursor that may induce an aberrant differentiation of the articular and entheseal chondrocytes near the osteochondral junctions, and the affected cells appeared to produce extracellular components including osteocalcin and type I collagen.

Exemplary cytoprotective non-steroidal anti-inflammatory compounds include for example sulindac. Sulindac ((Z)-5-fluoro-2-methyl-1-[[p-(methylsulfyl)phenyl]methylene]-1H-indene-3 acetic acid) also possessing analgesic and antipyretic activities. Its mode of action, like that of other non-steroidal, anti-inflammatory agents, is not known; however, its therapeutic action is not due to pituitary-adrenal stimulation.

Exemplary cytoprotective muscle relaxant compounds include succinylcholine chorlide, Succinylcholine Chloride (2,2′-[(1,4-dioxo-1,4-butanediyl)bis(oxy)bis [N,N,N-trimethylethanaminium]dichloride) is a skeletal muscle relaxant. It combines with the cholinergic receptors of the motor end plate to produce depolarization. Subsequent neuromuscular transmission is inhibited so long as adequate concentration of succinylcholine remains at the receptor site.

Exemplary cytoprotective dopaminergic agonist compounds, prolactin-inhibiting compounds such as bromocriptine. Bromocriptine (Ergotaman-3′,6′,18-trione,2-bromo-12′-hydroxy-2′-(1-methylethyl)-5′-(2-methylpropyl)-, (5′α) monomethanesulfonate) is used with levodopa/carbidopa to treat Parkinson's disease.

Exemplary cytoprotective carbonic anhydrase inhibitor compounds include methazolamide, acetazolamid, dorzolamide and brinzolamide compounds. Methazolamide (N-[5-(aminosulfonyl)-3-methyl-1,3,4-thiadiazol-2(3H)-ylidene]-acetamide), a sulfonamide is a potent inhibitor of carbonic anhydrase. Methazolamide decreases the secretion of aqueous humor and results in a decrease in intraocular pressure. a sulfonamide derivative; however, it does not have any clinically significant antimicrobial properties. Methazolamide achieves a high concentration in the cerebrospinal fluid.

Exemplary cytoprotective anesthetic compounds include corticosteroid compounds such as pramoxine, hydrocortizone, budesonide, pregnisone and cortizone. Pramoxine (4-[3-(4-Butoxyphenoxy)propyl]morpholine) is a topical anesthetic agent which provides temporary relief from itching and pain. It acts by stabilizing the neuronal membrane of nerve endings with which it comes into contact. Its unique chemical structure is likely to minimize the danger of cross-sensitivity reactions in patients allergic to other local anesthetics. Other exemplary anesthetic compounds include dyclonine hydrochloride. Dyclonine HCl (17-(Cyclopropylmethyl)-4,5-epoxy-3,14-dihydroxymorphinan-6-one) is a local anesthetic that blocks impulses at peripheral nerve endings in skin and mucous membranes by altering cell membrane permeability to ionic transfer.

Exemplary cytoprotective opioid antagonist compounds include naltrexone, propoxyphen and pentazocine. Naltrexone (17-(Cyclopropylmethyl)-4,5-epoxy-3,14-dihydroxymorphinan-6-one) is a synthetic congener of oxymorphone with no opioid agonist properties. Opioid antagonists have been shown to reduce alcohol consumption by animals, and naltrexone has been shown to reduce alcohol consumption in clinical studies.

Exemplary, cytoprotective thiol compounds include 2-mercaptoethanesulfonic acid, propyl mercaptan, ethyl mercaptan and butyl mercaptan.

Administration of Therapeutic Compositions

Effective doses vary, as recognized by those skilled in the art. Dosages for any one patient depends upon many factors, including the patient's size, body surface area, age, the particular compound to be administered, sex, time and route of administration, general health, and other drugs being administered concurrently.

Preferred unit dosage formulations are those containing an effective dose, as recited below, or an appropriate fraction thereof, of the active ingredient.

For each of the aforementioned conditions, the compositions may be administered orally or via injection at a dose of from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

A therapeutic regimen is carried out by identifying a mammal, e.g., a human patient suffering from (or at risk of developing) a neurological disorder using standard methods. Neurological disorders include, neurodegenerative disorders such as Amyotrophic Lateral Sclerosis, Alzheimer's disease, Huntington's disease and Parkinson's disease. Alternatively, the neurological disorder is a non-neurodegenerative disease such as diabetic neuropathy, cerebral hypoxia, encephalitis and menengitis For example, taurolidine or taurultam is administered to an individual diagnosed with a neurological disorder (e.g., acute myeloid leukemia) or an individual diagnosed with a precancerous condition (e.g., myelodysplasia which may progress to acute myeloid leukemia).

The pharmaceutical compound is to administered to such an individual using methods known in the art. Preferably, the compound is administered orally, topically or parenterally, e.g., subcutaneously, intraperitoneally, intramuscularly, and intravenously.

The compound is administered prophylactically, after the detection known risk factor, e.g., genetic or familial predisposition, attributed to the particular disease.

A cytoprotective compound is formulated into compositions for other routes of administration utilizing conventional methods. For example, it can be formulated in a capsule or a tablet for oral administration. Capsules may contain any standard pharmaceutically acceptable materials such as gelatin or cellulose. Tablets may be formulated in accordance with conventional procedures by compressing mixtures of a cytoprotective compound with a solid carrier and a lubricant. Examples of solid carriers include starch and sugar bentonite. The compound is administered in the form of a hard shell tablet or a capsule containing a binder, e.g., lactose or mannitol, a conventional filler, and a tableting agent. Other formulations include an ointment, paste, spray, patch, cream, gel, resorbable sponge, or foam. Such formulations are produced using methods well known in the art.

Alternatively, the compound is systemically administered or locally administered directly into CNS tissue. The compound is administered intravenously or intrathecally (i.e., by direct infusion into the cerebrospinal fluid). For local administration, a compound-impregnated wafer or resorbable sponge is placed in direct contact with CNS tissue. The compound or mixture of compounds is slowly released in vivo by diffusion of the drug from the wafer and erosion of the polymer matrix. A cytoprotective compound may be co administered with other know treatment regimes for a specific neurological disorder.

Alternatively, the compound is infused into the brain or cerebrospinal fluid using known methods. For example, a burr hole ring with a catheter for use as an injection port is positioned to engage the skull at a burr hole drilled into the skull. A fluid reservoir connected to the catheter is accessed by a needle or stylet inserted through a septum positioned over the top of the burr hole ring. A catheter assembly (e.g., an assembly described in U.S. Pat. No. 5,954,687) provides a fluid flow path suitable for the transfer of fluids to or from selected location at, near or within the brain to allow administration of the drug over a period of time.

Other embodiments are within the following claims.

What is claimed is:

1. A method of inhibiting death of a neuronal cell in a mammal, comprising administering to said mammal a composition comprising a steroid compound.

2. The method of claim 1, wherein said composition is administered at a dose sufficient to inhibit oxidative stress-induced neuronal cell death.

3. The method of claim 1, wherein said composition is administered at a dose sufficient to inhibit apoptotic death of said neuronal cell.

4. The method of claim 1, wherein said steroid compound is a progestin compound.

5. The method of claim 4, wherein said progestin compound is selected from the group consisting of (17α)-17-Hydroxy-19-norpregn-4-en-20-yn-3-one and 17a-(acetyloxy)-6-methylpregna-4,6-diene-3,20-dione.

6. The method of claim 1, wherein said steroid is an anti-inflammatory steroid.

7. The method of claim 6, wherein said anti-inflammatory steroid is flunisolide.

8. The method of claim 1, wherein said mammal is suffering from or at risk of developing a neurodegenerative disorder.

9. The method of claim 8, wherein said neurodegenerative disorder is selected from the group consisting of Amyotrophic Lateral Sclerosis, Alzheimer's disease, Huntington's disease and Parkinson's disease.

10. The method of claim 1, wherein said mammal is suffering from or at risk of developing a neurological disorder.

11. The method of claim 10, wherein said neurological disorder is selected from the group consisting of diabetic neuropathy, cerebral hypoxia, encephalitis and menengitis.

12. The method of claim 1, wherein said mammal is at risk of experiencing a stroke or has suffered a stroke.

13. A method of inhibiting death of a neuronal cell in a mammal, comprising administering to said mammal a composition comprising an anti-motion sickness agent.

14. The method of claim 13, wherein said composition is administered at a dose sufficient to inhibit oxidative stress-induced neuronal cell death.

15. The method of claim 13, wherein said composition is administered at a dose sufficient to inhibit apoptotic death of said neuronal cell.

16. The method of claim 13, wherein said anti-motion sickness agent is a HI histamine receptor blocker compound.

17. The method of claim 16, wherein said H1 histamine receptor blocker compound is 1-[(4-Chlorophenyl)phenylmethyl]-4-[(3-methylphenyl)methyl]piperazine.

18. The method of claim 13, wherein said anti-motion sickness agent is a belladonna alkaloid.

19. The method of claim 18, wherein said belladonna alkaloid is 6β,7β-epoxy-1αH,5αH-tropan-3α-ol(−)-tropate.

20. The method of claim 13, wherein said mammal is suffering from or at risk of developing a neurodegenerative disorder.

21. The method of claim 20, wherein said neurodegenerative disorder is selected from the group consisting of Amyotrophic Lateral Sclerosis, Alzheimer's disease, Huntington's disease and Parkinson's disease

22. The method of claim 13, wherein said mammal is suffering from or at risk of developing a neurological disorder.

23. The method of claim 22, wherein said neurological disorder is selected from the group consisting of diabetic neuropathy, cerebral hypoxia, encephalitis and menengitis.

24. The method of claim 13, wherein said mammal is at risk of experiencing a stroke or has suffered a stroke.

25. A method of inhibiting death of a neuronal cell in a mammal, comprising administering to said mammal a composition comprising an antibiotic compound at a dose sufficient to inhibit oxidative stress-induced neuronal cell death.

26. The method of claim 25, wherein said composition is administered at a dose sufficient to inhibit apoptotic death of said neuronal cell.

27. The method of claim 25, wherein said antibiotic compound is a macrolide antibiotic compound.

28. The method of claim 27, wherein said macrolide antibiotic compound is selected from the group consisting of erythromycin, troleandomycin, azithromycin and clarithromycin

29. The method of claim 25, wherein said antibiotic compound is a tetracycline compound or derivative.

30. The method of claim 29, wherein said tetracycline derivative compound is selected from the group consisting of chlorotetracycline, oxytetracycline, demeclocycline, methacycline. doxycycline and minocycline.

31. The method of claim 25, wherein said antibiotic is a tobramycin compound or a sulfacetamide compound.

32. The method of claim 25, wherein said mammal is suffering from or at risk of developing a neurodegenerative disorder.

33. The method of claim 32, wherein said neurodegenerative disorder is selected from the group consisting of Amyotrophic Lateral Sclerosis, Alzheimer's disease, Huntington's disease and Parkinson's disease.

34. The method of claim 25, wherein said mammal is suffering from or at risk of developing a neurological disorder.

35. The method of claim 34, wherein said neurological disorder is selected from the group consisting of diabetic neuropathy, cerebral hypoxia, encephalitis and menengitis.

36. The method of claim 25, wherein said mammal is at risk of experiencing a stroke or has suffered a stroke.

37. A method of inhibiting death of a neuronal cell in a mammal, comprising administering to said mammal a composition comprising a calcium channel blocker compound.

38. The method of claim 37, wherein said composition is administered at a dose sufficient to inhibit oxidative stress-induced neuronal cell death.

39. The method of claim 37, wherein said composition is administered at a dose sufficient to inhibit apoptotic death of said neuronal cell.

40. The method of claim 37, wherein said calcium channel blocker compound is selected from the group consisting of isopropyl (2-methoxyethyl) 1,4-dihydro-2,6-dimethyl-4-(3-nitrophenyl)-3,5-pyridine-dicarboxylate; α-[3-[[2-(3,4-dimethoxyphenyl)ethyl]methylamino]propyl]-3,4-dimethoxy-α-1(1-methylethyl)benzeneacetonitrile, 3,5-pyridinedicarboxylic acid; 1,4-dihydro-2,6-dimethyl-4-(2-nitrophenyl)-, dimethyl ester and 1,8-dihydroxy-9(10H)-anthracenone.

41. The method of claim 37, wherein said mammal is suffering from or at risk of developing a neurodegenerative disorder.

42. The method of claim 41, wherein said neurodegenerative disorder is selected from the group consisting of Amyotrophic Lateral Sclerosis, Alzheimer's disease, Huntington's disease and Parkinson's disease

43. The method of claim 37, wherein said mammal is suffering from or at risk of developing a neurological disorder.

44. The method of claim 43, wherein said neurological disorder is selected from the group consisting of diabetic neuropathy, cerebral hypoxia, encephalitis and menengitis.

45. The method of claim 37, wherein said mammal is at risk of experiencing a stroke or has suffered a stroke.

46. A method of inhibiting death of a neuronal cell in a mammal, comprising administering to said mammal a composition comprising an anti-depressant compound.

47. The method of claim 46, wherein said composition is administered at a dose sufficient to inhibit oxidative stress-induced neuronal cell death.

48. The method of claim 46, wherein said composition is administered at a dose sufficient to inhibit apoptotic death of said neuronal cell.

49. The method of claim 46, wherein said anti-depressant compound is selected from the group consisting of lithium carbonate, trazodone, bupropion hydrochloride, fluoxetine hydrocloride and sertraline hydrochloride.

50. The method of claim 46, wherein said mammal is suffering from or at risk of developing a neurodegenerative disorder.

51. The method of claim 50, wherein said neurodegenerative disorder is selected from the group consisting of Amyotrophic Lateral Sclerosis, Alzheimer's disease, Huntington's disease and Parkinson's disease

52. The method of claim 46, wherein said mammal is suffering from or at risk of developing a neurological disorder.

53. The method of claim 52, wherein said neurological disorder is selected from the group consisting of diabetic neuropathy, cerebral hypoxia, encephalitis and menengitis.

54. The method of claim 46, wherein said mammal is at risk of experiencing a stroke or has suffered a stroke.

55. A method of inhibiting death of a neuronal cell in a mammal, comprising administering to said mammal a composition comprising an alkali metal compound.

56. The method of claim 55, wherein said composition is administered at a dose sufficient to inhibit oxidative stress-induced neuronal cell death.

57. The method of claim 55, wherein said composition is administered at a dose sufficient to inhibit apoptotic death of said neuronal cell.

58. The method of claim 55, wherein said alkali metal compound is selected from the group consisting of lithium, caesium, rubidium and francium.

59. The method of claim 55, wherein said mammal is suffering from or at risk of developing a neurodegenerative disorder.

60. The method of claim 59, wherein said neurodegenerative disorder is selected from the group consisting of Amyotrophic Lateral Sclerosis, Alzheimer's disease, Huntington's disease and Parkinson's disease

61. The method of claim 55, wherein said mammal is suffering from or at risk of developing a neurological disorder.

62. The method of claim 61, wherein said neurological disorder is selected from the group consisting of diabetic neuropathy, cerebral hypoxia, encephalitis and menengitis.

63. The method of claim 55, wherein said mammal is at risk of experiencing a stroke or has suffered a stroke.

64. A method of inhibiting death of a neuronal cell in a mammal, comprising administering to said mammal a composition comprising an anti-arrhythmic agent.

65. The method of claim 64, wherein said composition is administered at a dose sufficient to inhibit oxidative stress-induced neuronal cell death.

66. The method of claim 64, wherein said composition is administered at a dose sufficient to inhibit apoptotic death of said neuronal cell.

67. The method of claim 64, wherein said anti-arrhythmic agent is a beta-adrenergic receptor blocking compound.

68. The method of claim 67, wherein said beta-adrenergic receptor blocking compound is selected from the group consisting of d, 1-N-[4-[1-hydroxy-2[(methylethyl)amino]ethyl]phenyl]methane-sulfonamide monohydrochloride and (S)-1-[(1,1-dimethylethyl)amino]-3-[[4-(4-morpholinyl)-1,2,5-thiadiazol-3-yl]oxy]-2-propanol (Z)-2-butenedioate

69. The method of claim 64, wherein said anti-arrhythmic agent is a sodium channel blocker compound.

70. The method of claim 69, wherein said sodium channel blocker compound is selected from the group consisting of lidocaine, mexiletine and prilocaine.

71. The method of claim 64, wherein said mammal is suffering from or at risk of developing a neurodegenerative disorder.

72. The method of claim 71, wherein said neurodegenerative disorder is selected from the group consisting of Amyotrophic Lateral Sclerosis, Alzheimer's disease, Huntington's disease and Parkinson's disease

73. The method of claim 64, wherein said mammal is suffering from or at risk of developing a neurological disorder.

74. The method of claim 73, wherein said neurological disorder is selected from the group consisting of diabetic neuropathy, cerebral hypoxia, encephalitis and menengitis.

75. The method of claim 64, wherein said mammal is at risk of experiencing a stroke or has suffered a stroke.

76. A method of inhibiting death of a neuronal cell in a mammal, comprising administering to said mammal a composition comprising dietary supplement at a dose sufficient to inhibit oxidative stress-induced neuronal cell death.

77. The method of claim 76, wherein said composition is administered at a dose sufficient to inhibit apoptotic death of said neuronal cell.

78. The method of claim 76, wherein said dietary supplement compound is selected from the group consisting of yohimbine, zinc, β-carotene, docosahexaenoic acid and retinol acetate.

79. The method of claim 76, wherein said dietary supplement compound is a presynaptic alpha-adrenergic receptor blocking compound.

80. The method of claim 79, wherein said presynaptic alpha-adrenergic receptor blocking compound is selected from the group consisting of yohimbine, medetomidine hydrochloride and atipamezole.

81. The method of claim 76, wherein said mammal is suffering from or at risk of developing a neurodegenerative disorder.

82. The method of claim 81, wherein said neurodegenerative disorder is selected from the group consisting of Amyotrophic Lateral Sclerosis, Alzheimer's disease, Huntington's disease and Parkinson's disease

83. The method of claim 76, wherein said mammal is suffering from or at risk of developing a neurological disorder.

84. The method of claim 83, wherein said neurological disorder is selected from the group consisting of diabetic neuropathy, cerebral hypoxia, encephalitis and menengitis.

85. The method of claim 76, wherein said mammal is at risk of experiencing a stroke or has suffered a stroke.

86. A method of inhibiting death of a neuronal cell in a mammal, comprising administering to said mammal a composition comprising muscle relaxant compound.

87. The method of claim 86, wherein said composition is administered at a dose sufficient to inhibit oxidative stress-induced neuronal cell death.

88. The method of claim 86, wherein said composition is administered at a dose sufficient to inhibit apoptotic death of said neuronal cell.

89. The method of claim 86, wherein said muscle relaxant compound is (Z)-5-fluoro-2-methyl-1-[[p-(methylsulfyl)phenyl]methylene]-1H-indene-3 acetic acid.

90. The method of claim 86, wherein said mammal is suffering from or at risk of developing a neurodegenerative disorder.

91. The method of claim 90, wherein said neurodegenerative disorder is selected from the group consisting of Amyotrophic Lateral Sclerosis, Alzheimer's disease, Huntington's disease and Parkinson's disease

92. The method of claim 86, wherein said mammal is suffering from or at risk of developing a neurological disorder.

93. The method of claim 92, wherein said neurological disorder is selected from the group consisting of diabetic neuropathy, cerebral hypoxia, encephalitis and menengitis.

94. The method of claim 86, wherein said mammal is at risk of experiencing a stroke or has suffered a stroke.

95. A method of inhibiting death of a neuronal cell in a mammal, comprising administering to said mammal a composition comprising a dopaminergic agonist compound.

96. The method of claim 95, wherein said composition is administered at a dose sufficient to inhibit oxidative stress-induced neuronal cell death.

97. The method of claim 95, wherein said composition is administered at a dose sufficient to inhibit apoptotic death of said neuronal cell.

98. The method of claim 95, wherein said dopaminergic agonist compound is prolatin-inhibiting compound.

99. The method of claim 95, wherein said prolatin inhibiting compound is bromocriptine.

100. The method of claim 95, wherein said mammal is suffering from or at risk of developing a neurodegenerative disorder.

101. The method of claim 100, wherein said neurodegenerative disorder is selected from the group consisting of Amyotrophic Lateral Sclerosis, Alzheimer's disease, Huntington's disease and Parkinson's disease

102. The method of claim 96, wherein said mammal is suffering from or at risk of developing a neurological disorder.

103. The method of claim 102, wherein said neurological disorder is selected from the group consisting of diabetic neuropathy, cerebral hypoxia, encephalitis and menengitis.

104. The method of claim 96, wherein said mammal is at risk of experiencing a stroke or has suffered a stroke.

105. A method of inhibiting death of a neuronal cell in a mammal, comprising administering to said mammal a composition comprising carbonic anhydrase inhibitor compound.

106. The method of claim 105, wherein said composition is administered at a dose sufficient to inhibit oxidative stress-induced neuronal cell death.

107. The method of claim 105, wherein said composition is administered at a dose sufficient to inhibit apoptotic death of said neuronal cell.

108. The method of claim 105, wherein said carbonic anhydrase inhibitor compound is selected from the group consisting of methazolamide, acetazolamide, dorzolamide and brinzolamide.

109. The method of claim 105, wherein said mammal is suffering from or at risk of developing a neurodegenerative disorder.

110. The method of claim 109, wherein said neurodegenerative disorder is selected from the group consisting of Amyotrophic Lateral Sclerosis, Alzheimer's disease, Huntington's disease and Parkinson's disease

111. The method of claim 105, wherein said mammal is suffering from or at risk of developing a neurological disorder.

112. The method of claim 111, wherein said neurological disorder is selected from the group consisting of diabetic neuropathy, cerebral hypoxia, encephalitis and menengitis.

113. The method of claim 105, wherein said mammal is at risk of experiencing a stroke or has suffered a stroke.

114. A method of inhibiting death of a neuronal cell in a mammal, comprising administering to said mammal a composition comprising an anesthetic compound.

115. The method of claim 114, wherein said composition is administered at a dose sufficient to inhibit oxidative stress-induced neuronal cell death.

116. The method of claim 114, wherein said composition is administered at a dose sufficient to inhibit apoptotic death of said neuronal cell.

117. The method of claim 114, wherein said anesthetic compound is corticosteroid compound.

118. The method of claim 117, wherein said corticosteroid compound is selected from the group consisting of pramoxine, hydocortizone, hetamethazone, budesonide, prednisone and cortisone.

119. The method of claim 114, wherein said anesthetic is dyclonine hydrochoride.

120. The method of claim 114, wherein said mammal is suffering from or at risk of developing a neurodegenerative disorder.

121. The method of claim 120, wherein said neurodegenerative disorder is selected from the group consisting of Amyotrophic Lateral Sclerosis, Alzheimer's disease, Huntington's disease and Parkinson's disease

122. The method of claim 114, wherein said mammal is suffering from or at risk of developing a neurological disorder.

123. The method of claim 122, wherein said neurological disorder is selected from the group consisting of diabetic neuropathy, cerebral hypoxia, encephalitis and menengitis.

124. The method of claim 114, wherein said mammal is at risk of experiencing a stroke or has suffered a stroke.

125. A method of inhibiting death of a neuronal cell in a mammal, comprising administering to said mammal a composition comprising an opioid antagonist compound.

126. The method of claim 125, wherein said composition is administered at a dose sufficient to inhibit oxidative stress-induced neuronal cell death.

127. The method of claim 125, wherein said composition is administered at a dose sufficient to inhibit apoptotic death of said neuronal cell.

128. The method of claim 125, wherein said opiod antagonist compound is selected from the group consisting naltrexone, propoxyphene and pentazocine.

129. The method of claim 125, wherein said mammal is suffering from or at risk of developing a neurodegenerative disorder.

130. The method of claim 129, wherein said neurodegenerative disorder is selected from the group consisting of Amyotrophic Lateral Sclerosis, Alzheimer's disease, Huntington's disease and Parkinson's disease

131. The method of claim 125, wherein said mammal is suffering from or at risk of developing a neurological disorder.

132. The method of claim 131, wherein said neurological disorder is selected from the group consisting of diabetic neuropathy, cerebral hypoxia, encephalitis and menengitis.

133. The method of claim 125, wherein said mammal is at risk of experiencing a stroke or has suffered a stroke.

134. A method of inhibiting death of a neuronal cell in a mammal, comprising administering to said mammal a composition comprising a thiol compound.

135. The method of claim 134, wherein said composition is administered at a dose sufficient to inhibit oxidative stress-induced neuronal cell death.

136. The method of claim 134, wherein said composition is administered at a dose sufficient to inhibit apoptotic death of said neuronal cell.

137. The method of claim 134, wherein said thiol compound is selected from the group consisting 2-mercaptoethanesulfonic acid, propyl mercaptan, ethyl mercaptan and butyl mercaptan.

138. The method of claim 134, wherein said mammal is suffering from or at risk of developing a neurodegenerative disorder.

139. The method of claim 138, wherein said neurodegenerative disorder is selected from the group consisting of Amyotrophic Lateral Sclerosis, Alzheimer's disease, Huntington's disease and Parkinson's disease

140. The method of claim 134, wherein said mammal is suffering from or at risk of developing a neurological disorder.

141. The method of claim 140, wherein said neurological disorder is selected from the group consisting of diabetic neuropathy, cerebral hypoxia, encephalitis and menengitis.

142. The method of claim 134, wherein said mammal is at risk of experiencing a stroke or has suffered a stroke.

143. A method of inhibiting death of a neuronal cell in a mammal, comprising administering to said mammal a composition comprising a non-steroidal anti-inflammatory compound.

144. The method of claim 143, wherein said composition is administered at a dose sufficient to inhibit oxidative stress-induced neuronal cell death.

145. The method of claim 143, wherein said composition is administered at a dose sufficient to inhibit apoptotic death of said neuronal cell.

146. The method of claim 143, wherein said non-steroidal anti-inflammatory compound is selected from the group consisting sulindac, ibuprofen, nabumentone, naproxen and acetaminophen.

147. The method of claim 143, wherein said mammal is suffering from or at risk of developing a neurodegenerative disorder.

148. The method of claim 147, wherein said neurodegenerative disorder is selected from the group consisting of Amyotrophic Lateral Sclerosis, Alzheimer's disease, Huntington's disease and Parkinson's disease

149. The method of claim 143, wherein said mammal is suffering from or at risk of developing a neurological disorder.

150. The method of claim 149, wherein said neurological disorder is selected from the group consisting of diabetic neuropathy, cerebral hypoxia, encephalitis and menengitis.

151. The method of claim 143, wherein said mammal is at risk of experiencing a stroke or has suffered a stroke.
